(19) United States
(12) Patent Application Publication
(10) Pub. No.: US 2008/0070281 A1
(43) Pub. Date: Mar. 20, 2008

(54) Method for Stabilising Reagents Which are Useful for Nucleic Acid Amplification

(76) Inventors: Peter John White, Salisbury (GB); Mark Basche, Salisbury (GB)

Correspondence Address: NIXON & VANDERHYE, PC, 901 NORTH GLEBE ROAD, 11TH FLOOR, ARLINGTON, VA, 22203, US

(21) Appl. No.: 11/630,645

(22) PCT Filed: Jul. 4, 2005
(86) PCT No.: PCT/GB05/02628

§ 371 Date: Mar. 8, 2007

(30) Foreign Application Priority Data
Jul. 2, 2004 (GB) 0414815.1

(51) Int. Cl. C12P 19/34; C12M 1/40; C12N 9/96

(52) U.S. Cl. 435/91.2; 435/188; 435/289.1

(57) ABSTRACT

A method for stabilising reagents suitable for use in a nucleic acid amplification reaction has now been developed comprising: (i) preparing a reagent mixture comprising reagents suitable for use in a nucleic acid amplification reaction wherein the mixture comprises a polynucleotide polymerase; and (ii) drying the reagents; characterised in that said reagent mixture comprises from about 0.1% to about 50% of the final concentration of magnesium ions required to activate an amplification reaction. Reagents, reaction vessels, use of such reagents in a nucleic acid amplification reaction, and a method of conducting an amplification reaction using reagents so prepared are also disclosed herein.

### FIELD OF THE INVENTION

This invention relates to a method for the stabilisation of reagents, particularly reagents to be used in a nucleic acid amplification reaction. This invention also relates to stabilised reagents, reaction vessels comprising such reagents and use of such reagents.

### BACKGROUND

Some reagents are not stable at ambient temperature, pressure and humidity. In the controlled environment of a laboratory their stability can be readily managed, for example by storing reagents at reduced temperatures or storing reagents in oxygen free atmospheres, but the stable storage such reagents used outside of a laboratory environment is more difficult. Furthermore, many procedures require complex mixtures of reagents. Again, in the laboratory such reagents can be stored separately until required to prevent degradation or side reactions. But when developing procedures for use outside of a laboratory environment by a worker with little or no scientific training it is preferable to develop ways in which reagents can be pre-mixed and stored without degradation or side reactions, to simplify the required procedure. As such, innovative solutions are required to stabilise different types of reagents and mixtures of such to allow them to be successfully stored and used in a wide variety of environments and instrument platforms.

One example of a laboratory procedure that is currently being developed for use outside of the laboratory is nucleic acid amplification reactions. These reactions, which amplify a wide variety of different nucleic acid targets, are well known and are routinely performed in laboratories. An example of such an amplification reaction is the polymerase chain reaction (PCR). The usefulness of this reaction in diagnosing disease states, identifying contaminants in the environment or food, as a tool for forensic science, clinical microbiology, oncology, blood banking is well known. However, to date, it has been necessary to use laboratory based protocols to conduct such reactions due to their complexity, the inherent stability of the reagents, the possibility of side reactions when reagents are first mixed and the expertise and equipment required. Recently progress has been made towards developing equipment and procedures that can be used to conduct nucleic acid amplification reactions outside of the laboratory, for example in the field or in a clinic, by workers with little or no scientific training. Such a system would allow for the completion of individual tests to provide rapid sample identification soon after collection.

Nucleic acid amplification reactions require many different reagents. Core reagents include an amplification enzyme for example a polynucleotide polymerase for example a thermostable polymerase, nucleoside triphosphates, oligonucleotide primers that are complementary to the target material, magnesium ions and other buffers. Furthermore, assay formulations used in real time PCR or qPCR will also use reagents that can include dye labelled oligonucleotide probes, DNA binding dyes e.g. Sybr Gold and internal control DNAs.

There is a need for new approaches to be developed to create reagent formulations with a good shelf life and excellent performance for non-laboratory based nucleic amplification systems. This would ensure that the reagents have an adequate shelf life and minimise degradation that could lead to test failure or receipt of a false positive result. To further simplify the procedure it is desirable that as many as possible of the required reagents are mixed, prior to storage, in the required quantities. However it is important that after such reagents have been mixed, and during storage, side reactions are minimised. In particular, pre-combination of the nucleic acid amplification reagents may lead to premature mis-primed amplification of nucleotide sequences within the mixture due to non specific annealing of primers ahead of the addition of the target material even if the formulation is prepared at low temperatures (0-4° C.). This can lead to failure of the target amplification since unwanted artefacts are generated that may interfere with the amplification and/or target detection, especially when low copy number amplifications are performed. Furthermore pre-combination of the reagents and storage in solution may lead to degradation of the reagents over time. Although this can be solved in part by storing the reagents in a dry powder form, for example freeze drying, the problems are not obviated completely. This is because during formulation and prior to freeze drying some reagent degradation/mis-primed amplification may occur, and if the equipment is to be stored or used in humid conditions reagent rehydration may occur. Furthermore, the freeze drying process itself may promote undesirable interactions between reaction components during the transition from the liquid to the glass state as the concentration of the reagents increases. As such there is a need for new and improved formulations and stabilisation systems that allow for long term storage of pre-mixed reagents, including those for use in nucleic acid amplification reactions, at ambient temperatures ideally for at least 3 months.

Some work has already been conducted into stabilising reagents prior to nucleic acid amplification. For example Setterquist et al (Nucleic Acids Research 1996, vol 24 pp 1580-1581) discloses a method for encapsulating the components of a PCR reaction in a matrix comprising 0.5% agarose/50% glycerol that can be readily shipped, even at ambient temperature, and stored at −20° C. for many months. The PCR reaction can be initiated simply by adding the target DNA in solution and thermocycling the mixture. However these mixtures are not suitable for storage at ambient temperatures. Alternatively U.S. Pat. No. 5,599,660 discloses a method for the storage and delivery of reagents, optionally for a PCR reaction, comprising encapsulating a first reagent in a wax carrier and combining this with a second reagent, optionally stored in a glassy or dehydrated form. The two reagents are then mixed by dissolving the wax with a suitable solvent or heating the wax until it melts.

The prior art also includes a number of suggestions to stabilise the reaction mixture by eliminating one of the key amplification reagents and adding this immediately prior to amplification. For example Kaijalainen et al (Nucleic Acids Research 1993, vol 21, pp 2959-2960) discloses a method of stabilising a PCR reaction mixture by drying and embedding the primers within a wax bead so that they are released into remainder of the amplification mixture as it is heated and the wax melts. Blair et al (PCR Methods and Applications 1994, vol 4 pp 191-194) discloses cosolidifying the PCR reagents, including non thermostable reagents, with wax but omitting either the primer or the thermostable enzyme, which is then added as a solution immediately prior to amplification. However in each of these cases, if the reaction is to be conducted in a non-laboratory environment, it is still necessary for the non-skilled worker to add a critical reagent in the correct amount prior to amplification. Furthermore, in some of these mixtures mis-priming events still occurred resulting in side reactions and unwanted artefacts.

The stabilisation of reagents for amplification by removal of magnesium ions from the reaction mixture has also been disclosed. This has the advantage that in the absence of magnesium the polymerase is inactive. U.S. Pat. No. 5,411,876 discloses formulating the reagents as two subsets, the first comprising magnesium and the second comprising all other reagents, and separating the subsets within the reaction vessel by a layer of wax/grease optionally comprising a surfactant. U.S. Pat. No. 6,403,341 discloses sequestering the magnesium, optionally with a source of phosphate ions, as a precipitate which is able to dissolve at elevated temperatures, adding the remainder of the reagents and allowing the reagents to mix when thermocycling begins. The prior art teaches that it is important that, since the polymerase is stored in the presence of primers and triphosphates that the reaction mixture does not comprise any magnesium otherwise mis-priming may still occur. In order to ensure that no free magnesium is present magnesium sequestering materials such as chelators are added. However, it has now been observed that in some such systems the reagents are not completely stabilised during storage and the formation of unwanted artefacts is still observed. There remains a need to develop a further improved method of stabilising reagents, particularly those for use in a nucleic acid amplification reaction, for storage.

A new and improved method for stabilising reagents suitable for use in a nucleic acid amplification reaction has now been developed. The method comprises:
(i) preparing a reagent mixture comprising reagents suitable for use in a nucleic acid amplification reaction wherein the mixture comprises a polynucleotide polymerase; and
(ii) drying the reagents;
   characterised in that said reagent mixture comprises from about 0.1% to about 50% of the final concentration of magnesium ions required to activate an amplification reaction.

The presence of magnesium is believed to affect the amplification reaction by the following mechanisms: activating the polynucleotide polymerase enzyme, interacting with the oligonucleotides, complexing with the dNTP's and buffering the reaction mixture.

The determination of the final concentration of magnesium ion required to activate an amplification reaction is known to the art as requiring trial and error, there being an optimum range, for a particular polynucleotide polymerase, in which the reaction proceeds with the desired specificity. The final concentration of magnesium ion required to activate a desired amplication reaction may typically range between 1 mM and 5 mM.

When the level of magnesium is chosen such that the amplification does not proceed mis-priming events are minimised or prevented. It is further believed that by formulating the reaction mixture to comprise some magnesium ions unfavourable interactions between reaction components, in particular oligonucleotide primers, probes and DNA binding dyes, that can occur during the freeze drying process is minimised thereby ensuring primers and probes are available to bind to the target. This improves the efficiency of the amplification reaction and reduces the formation of side products or unwanted artefacts during storage or amplification.

The step of drying the reagent mixture stabilises the formulation for room temperature storage minimising reagent degradation. A reagent mixture so stabilised can be used in a nucleic acid amplification by the addition of a suitable solvent comprising the remainder of the required magnesium ions and the target to be amplified.

The method is optionally improved by the additional step of separating the dried reagent mixture from the atmosphere by a layer of wax or grease. When the dried reagents are reconstituted by addition of solvent, target material and remaining magnesium ions is added it initially remains separated from the dried reagents by the layer of wax or grease. This ensures that no mixing of the target material or magnesium ions with the polymerase occurs until the wax or grease begins to melt as a result of heating the reaction mixture. This further minimises mis-priming reactions that lead to side products or unwanted artefacts. If the wax or grease has a lower density than water upon melting it will form a top layer above the reaction mixture. This has the additional advantage that solvent is prevented from evaporating during thermocycling, which is important since these reactions are usually conducted in very small volumes. Furthermore, after the amplification reaction is completed, the wax/grease will solidify on top of the reaction mixture as it cools. This seals the reagents and amplified target allowing for safe disposal without fear of the amplified target contaminating the user or further reactions.

This method of the present invention has several advantages including that it provides an improved method for the stabilisation of reagents suitable for use in a nucleic acid amplification reaction. It allows for pre-mixed reagents to be sufficiently stabilised to allow for storage in a non-laboratory environment at ambient temperature over a period of time, ideally a minimum of 3 months at 25° C. Furthermore the stabilisation method minimises the formation of reaction artefacts either prior to or during amplification thereby improving the amplification efficiency and detection of the target material. This is especially useful where the target material is available in low concentration or has a low copy number.

This invention also relates to reagents that have been stabilised according to the method of the present invention and also to a reaction vessel comprising reagents that have been stabilised according to the present invention. The advantage of stabilising the reagents according to the present invention directly into a reaction vessel suitable for use directly in the nucleic acid amplification reaction is that the reagents do not need to be transferred into a reaction vessel prior to use. In a non-laboratory environment this removes the requirement of needing to measure out the required amount of reagent thereby simplifying the process. It also reduces the possibility of the reagents or reaction vessel becoming contaminated during use.

This invention also relates to a method for stabilising reagents suitable for use in a nucleic acid amplification reaction comprising:
(i) preparing a reagent mixture comprising reagents suitable for use in a nucleic acid amplification reaction wherein the mixture comprises a polynucleotide polymerase;
(ii) drying the reagents; and
(iii) covering the dried reagents with a layer of wax or grease;
   characterised in that the reagent mixture comprises insufficient magnesium ions to activate an amplification reaction.

This method has several advantages including that it provides an improved method for the stabilisation of reagents suitable for use in a nucleic acid amplification reaction. It allows for pre-mixed reagents to be sufficiently stabilised to allow for storage in a non-laboratory environment at ambient temperature over a period of time, ideally a minimum of 3 months at 25° C. The addition of a covering of wax or grease over the dried reagents minimises any rehydration of reagents that may occur during storage of the reagents. This is particularly useful if the reagents are to be stored in damp or humid environments. Furthermore by ensuring that the reaction mixture comprises insufficient magnesium ions to activate the amplification reaction, thereby ensuring minimal activity of the polynucleotide polymerase, the formation of artefacts in the reaction mixture either prior to or during amplification is minimised thereby improving the amplification efficiency and subsequent detection of the target material. This invention also relates to reagents so stabilised and reaction vessels suitable for use in a nucleic acid amplification reaction comprising reagents so stabilised.

It is an object of this invention to develop a method to allow for the stable storage of reagents suitable for use in a nucleic acid amplification reaction at ambient temperatures. This method should minimise side reactions that may occur either prior to or during amplification reaction thereby reducing unwanted artefacts and increasing the efficiency of the amplification reaction. Even further this method should minimise unfavourable interactions between reaction components during the drying process thereby further minimising the formation of unwanted artefacts. It is a further object of this invention to develop reagents so stabilised and reaction vessels comprising reagents so stabilised. These and other objects of the present invention will become apparent in light of the following disclosure.

### SUMMARY OF THE INVENTION

According to a first aspect this invention relates to a method for stabilising reagents suitable for use in a nucleic acid amplification reaction has now been developed comprising:
(i) preparing a reagent mixture comprising reagents suitable for use in a nucleic acid amplification reaction wherein the mixture comprises a polynucleotide polymerase; and
(ii) drying the reagents;
   characterised in that said reagent mixture comprises from about 0.1% to about 50% of the final concentration of magnesium ions required to activate an amplification reaction.

According to a second aspect this invention relates to reagents suitable for use in a nucleic acid amplification reaction stabilised according to the present invention.

According to a third aspect this invention relates to reaction vessels suitable for use in a nucleic acid amplification reaction comprising reagents stabilised according to the present invention.

According to a fourth aspect this invention relates to the use of reagents so stabilised in a nucleic acid amplification reaction.

According to a fifth aspect this invention relates to a method of performing a nucleic acid amplification reaction comprising:
(i) preparing a reagent mixture according to the present invention;
(ii) adding to said reagent mixture the target material to be amplified, sufficient further magnesium ions to activate the amplification reaction and a suitable solvent; and
(iii) heating and cooling the so formed reaction mixture.

According to a sixth aspect this invention relates to a method for stabilising reagents suitable for use in a nucleic acid amplification reaction comprising:
(i) preparing a reagent mixture comprising reagents suitable for use in a nucleic acid amplification reaction wherein the mixture comprises a polynucleotide polymerase;
(ii) drying the reagents; and
(iii) covering the dried reagents with a layer of wax or grease;
   characterised in that the reagent mixture comprises insufficient magnesium ions to activate an amplification reaction.

### DESCRIPTION

All publications cited herein are hereby incorporated by reference in their entirety, unless otherwise indicated.

As used herein the term “reagent” shall refer to any substance that could be the component of in a chemical or biochemical reaction, particularly a nucleic acid amplification reaction, such as enzymes, peptide hormones, structural proteins, amino acids, antibodies, molecules containing protein groups, RNA, DNA, nucleic acids, primers, probes, buffers and proteins conjugated to nucleic acids. A reagent could also be a detection substance including probes to which fluorophores have been attached, nucleic acid intercalating dyes such as DNA binding dyes for example ethidium bromide, Sybr Gold and the like.

As used herein the term “magnesium ions” shall refer to any substance containing magnesium in the form such that divalent magnesium is released into any aqueous solvent preferably with a pH of from about 6 to about 9. Possible substances that are able to release magnesium ions include but are not limited to magnesium chloride, magnesium hydroxide, magnesium carbonate and magnesium sulphate.

As used herein the term “nucleic acid reaction vessel” shall refer to any container suitable for holding nucleic acid amplification reagents during an amplification and therefore should not be made of a material that inhibits such a reaction. Commonly such vessels are manufactured from polypropylene. The material from which the reaction vessel is made should be selected such that it is able to withstand temperatures in a range of from about 20° C. to about 100° C. while retaining substantially the same size/shape and can be capable of completing a change in the temperature of the contents of about 40° C. when effected over a time period of not more than about 4 minutes.

As used herein the term “oil” shall refer to a water immiscible organic substance, liquid at temperatures less than about 40° C. and which has a lower density than water. “Mineral oil” also known as liquid petroleum and paraffin oil, is a colourless, optically clear mixture of high-molecular either hydrocarbons with a density of near 0.84 g/ml, widely available commercially and commonly used as a vapour barrier over nucleic acid amplification reactions.

As used herein the term “wax” refers to any group of substances composed of hydrocarbons, alcohols, fatty acids and esters that are solid at ambient temperature. These substances may be of plant or animal origin and contain principally esters of higher fatty acids and higher alcohols, free fatty acids and alcohols, and saturated hydrocarbons. A suitable carrier wax will be liquid at certain temperature and solid at a lower temperature. Additionally a suitable wax will not be soluble or swellable in an aqueous solution. Preferably the carrier wax is selected from material that has a melting point above room temperature. Most preferably, the carrier wax is selected from material that has a melting point above 37° C. so that at normal variations of room temperature the co-solidified material remains solid. When melted the wax preferably forms a liquid that has a lower density than water. Typical pure compounds that are useful waxes include eicosane, octacosane, cetyl palmitate and pentaerythritol, tetrabehenate. Typical wax mixtures include but are not limited to, paraffin, paraplast, ultraflex and Besquare 175, Ampliwax (Perkin Elmer Cetus) and Polyfin (Polysciences). Waxes can be prepared by mixing pure or mixed waxes with one another or with greases or oils in any ratios which preserve the characteristic of a wax in general. Such techniques are well known to one skilled in the art.

As used herein the term “grease” shall refer to an organic substance, solid or semi-solid but very soft at temperatures below about 40° C., which melts in the range of from about 40° C. to about 80° C. to form a liquid that has a lover density than water. A typical grease is white petroleum, a mixture of high molecular weight hydrocarbons.

As used herein the term “surfactant” shall mean a substance that reduces the interfacial tension between water or aqueous solutions and hydrophobic solids or liquids like polyolefin plastics, oils, greases, and waxes. Surfactants are composed structurally of covalently joined hydrophilic and hydrophobic moieties. “Non-ionic surfactants” contain no positively or negatively charged moieties. Typical non ionic surfactants include the following families of structural homologues: Span, Tween, Brij, Myrj and Triton.

Dehydrated and freeze dried biological and chemical reagents can be prepared according to the methods described in among others L. R. Rey “Glimpses into the Fundamental Aspects of Freeze Drying” in International Symposium on Freeze Drying of Biological products Washington D.C. 1976 in Develop. Biol. Standard 36: 19-27, 1977 (S. Karger, Basel). Alternatively the material may be preserved in a “glass” made of polysaccharides such as described in U.S. Pat. No. 5,250,429 and U.S. Pat. No. 5,098,893. In both cases water or aqueous solvent is generally added to rehydrate the stabilised reagents.

The present invention relates to a method for stabilising reagents suitable for use in a nucleic acid amplification reaction has now been developed comprising:
(i) preparing a reagent mixture comprising reagents suitable for use in a nucleic acid amplification reaction wherein the mixture comprises a polynucleotide polymerase; and
(ii) drying the reagents;
   characterised in that said reagent mixture comprises from about 0.1% to about 50% of the final concentration of magnesium ions required to activate an amplification reaction.

Reagents that are commonly mixed for use in a nucleic acid amplification reaction include those selected from the following: all four compound nucleoside triphosphates (eg for DNA polymerase the four common dNTP's—dATP, dGTP, dTTP, dCTP) at a concentration in the range of about 1×10⁻⁵M to about 1×10⁻³M; magnesium ions in the form of a suitable substance, usually MgCl₂, usually at concentrations of about 1-5 mM; a polynucleotide polymerase, preferably a thermostable polymerase, more preferably a thermostable DNA polymerase, most preferably the DNA polymerase I from Thermus aquaticus (Taq polymerase, as described in U.S. Pat. No. 4,889,818), usually at a concentration of from about 1×10⁻¹⁰M to about 1×10⁻⁸M; and single stranded oligonucleotide primers containing base sequences which are complementary to sequences on both strands of the target nucleic acid sequence usually is present at a concentration of about 1×10⁻⁷M to about 1×10⁻⁵M. The primers are generally synthesised by solid phase methods well known in the art of nucleic acid chemistry.

The nucleic acid amplification reaction occurs when a target nucleic acid that is to be amplified is added to a solution comprising the above reagents. The mixture is then cyclically heated during which the amplification can occur. The amplification reaction is usually conducted in approximately about 5 to about 200 μl of solvent, preferably aqueous solution buffered to have a pH in the range of from about 6 to about 9.

Optionally the amplification reaction mixture may also comprise labelled oligonucleotide probes which may optionally be labelled with a dye, including fluorescent dyes; nucleic acid intercalating dyes which may optionally be fluorescent and including DNA binding fluorescent dyes for example ethidium bromide, SYBR Gold and the like; bovine serum albumin; internal control nucleic acid and mixtures thereof.

In the present invention the desired reagents are mixed together. Preferably the reagents are those necessary for a nucleic acid amplification reaction, more preferably comprise a thermostable polymerase and even more preferably do not comprise the target nucleic acid which it is intended to amplify during the reaction. In order to minimise the reaction between reagents during the mixing process it is preferred that they are mixed at a temperature of less than about 15° C., more preferably less than about 10° C. and most preferably less than about 5° C.

After the reagents have been mixed together they are dried to remove any solvent, usually aqueous solvent. The removal of solvent provides a first aspect of the stabilisation procedure allowing the reagents to be stored in this form at ambient temperature for a period of time. The reagent mixture can be dried by any method known in the art. Preferably the method is chosen to prevent or minimise side reactions occurring in the reagent mixture and therefore ideally does not comprise heating the reagent mixture to high temperatures. The reagent mixture is preferably dried using freeze drying methods or alternatively air drying methods such as lyophilisation that are known to those skilled in the art. When conducting such a drying method saccharides, such as trehlaose, may optionally be added to the reagent mix to stabilise the protein components.

The reagent mixture comprises about 0.1% to about 50%, preferably from about 3% to about 30% and more preferably from about 5% to about 15% of the final concentration of magnesium ions necessary to activate an amplification reaction. Optionally the level of magnesium ions chosen is from about 0.1% to about 50%, preferably from about 3% to about 30% and more preferably from about 5% to about 15% of the final concentration of magnesium ions necessary to activate the polynucleotide polymerase.

Magnesium ions are thought to have several key roles in amplification reactions. These include activating the polynucleotide polymerase enzyme, interacting with the oligonucleotides, complexing with the dNTP's and buffering the reaction mixture. The availability of magnesium ions will therefore be affected by many factors well known to those skilled in the art including the concentration of dNTP's used, the concentration of oligonucleotides used and the like. The availability of magnesium ions may also be affected by other factors including the material from which the reaction vessel is made. However if insufficient magnesium ions are available the amplification reaction will not proceed. It is therefore necessary to optimise the final amplification reaction mixture to ascertain the amount of magnesium required in order for the amplification to proceed. This can be readily conducted by one of ordinary skill in the art. Such optimisation will include identifying the level of magnesium required in order to activate the polynucleotide polymerase enzyme.

It is important that the level of magnesium ions are insufficient to activate the amplification reaction or the polynucleotide polymerase such that mis-priming events are prevented when the mixture is initially prepared prior to addition of the target. However, it has now been shown that the inclusion of some magnesium further minimises the production of unwanted artefacts when the reagent mixture is later reconstituted for use in a nucleic acid amplification reaction. Without wishing to be bound by theory it is believed that this is because the inclusion of a small amount of magnesium in the reagent mixture is minimises unfavourable interactions between reaction components during the freeze drying process. It is believed that as a result the formation of very close interactions between the oligonucleotide primers/probes and the enzyme is minimised. This has the result that during a later amplification a reduction of unwanted artefacts is observed. Overall this inclusion of a low amount of magnesium prior to drying has the effect of further stabilising the formulation and optimising it for further use in an amplification reaction.

The reagent mixture may, depending on a particular polynucleotide polymerase, comprises magnesium ion at a concentration of from about 0.1 mM to about 10 mM, from about 0.5 mM to about 5 mM or from about 1 mM to about 2.5 mM. Preferably, however, the reagent mixture comprises magnesium ions at a concentration below 500 μM. The magnesium ion concentration may, in particular, and especially where a Taq polymerase is used, range between 10 μM and 300 μM and preferably between 10 μM and 100 μM.

The term “activate the amplification reaction” means that when an amplification reaction is conducted using a given level of magnesium ions using standard amplification thermocycling conditions amplification products are detected. Such products may or may not be amplification of the desired target material. Alternatively they may relate to amplification of other components of the reaction mixture for example unwanted amplification of oligonucleotide primers and the like. Such amplification products can be detected by any one of a wide range of suitable methods known to those skilled in the art. When the amplification reaction is not activated only a minimal level, and preferably no, amplification products will be observed. The standard amplification thermocycling conditions and detection conditions will vary depending on the type of amplification reaction that is being conducted but will be well known to those skilled in the art. If the amplification products are detected using fluorescence then when the amplification reaction is not active only minimal or preferably no fluorescence indicative of an amplification product will be detected.

The term “activate the polynucleotide polymerase” means that when an amplification reaction is completed using standard amplification thermocycling conditions with the chosen level of magnesium that amplification products are detected. Such products can be detected by any one of a wide range of suitable methods known to those skilled in the art. When the polynucleotide polymerase is not active only a minimal level, and preferably no, amplification products will be observed. The standard amplification thermocycling conditions and detection conditions will vary depending on the type of amplification reaction that is being conducted but will be well known to those skilled in the art. If the amplification products are detected using fluorescence then when the polynucleotide polymerase is not active only minimal or preferably no fluorescence indicative of an amplification product will be detected.

Optionally the method of the present invention may include the additional step of covering the dried reagents with a layer of wax or grease. If the reagents are stored within a container this may mean providing a sealing layer within the container above the dried reagents. Alternatively this may mean encapsulating the dried reagents within a vesicle which is manufactured from wax or grease. The amount of wax or grease used should preferably be sufficient to form a barrier between the dried reagent mixture and the atmosphere. This barrier further increases the stabilisation of the dried reagent mixture thereby increasing the shelf life of the dried reagents at ambient conditions. The layer may be prepared such that the wax or grease is in contact with the reagents. Alternatively the layer may be such that the wax or grease forms a plug within a vessel in which the dried reagents are stored. Other suitable ways of applying the wax or grease layer may also be determined by one skilled in the art such as forming a vesicle in which the dried reagents may be stored and the like.

Any wax, greases or oils or mixtures thereof known in the art may be used. It is preferred that the wax, grease or oil is solid or viscous at room temperature thereby forming a protective layer which separates the reagents from the atmosphere and which does not leak out of any container even during shipping. It is most preferred to use a wax since this is most able to effectively form a barrier. Preferably the material melts in the range of from about 40° C. to about 90° C. Preferably when melted the material has a density of less than water such that it floats to the top of the reaction mixture when the dried reagents are reconstituted with an aqueous solvent. Optionally the wax or grease may comprise a surfactant that reduces the depth of the meniscus between the wax or grease and the water thereby reducing the mass of wax or grease needed to completely cover the solubilised reaction mixture during amplification.

If necessary the wax or grease layer may be thinned by incorporation into it of polymeric particles or of relatively fine plastic mesh. Examples of suitable plastics include but are not limited to polyethylene, polypropylene, polymethylpentene, polyester, nylon and various fluorocarbons. It is preferred that any plastics chosen are not able to bind the reagents for the amplification reaction, particularly nucleic acid sequences. Examples of suitable polymeric particles include but are not limited to polystyrene, polymethylmethacrylate. They can be spherical or irregular in shape. Non porous materials are preferred since they offer a lower surface area to entrap reagents. Preferably the particles have a density of less than or very close to water such that they are likely to form a layer on top of the aqueous layer when the former melts into an oil. The concentration of polymeric particles in the grease or wax permits considerable variability and can be optimised for any of several functional properties of the mixture as known to one skilled in the art.

It is preferred that the reagents are placed into a container in which they are to be dried as soon as possible, preferably that the reagents are mixed directly in the container in which they are to be dried. Furthermore it is preferred that the reagents are dried directly within the vessel in which they will be ultimately utilised for a reaction, for example a nucleic acid amplification reaction vessel. This minimises the chances of contamination of the reagents prior to use as they are transferred into the reaction vessel. Furthermore it means that the desired amount of reagent can be directly measured into the reaction vessel thereby simplifying the use of the vessel in the field.

According to a further aspect this invention relates to reagents, particularly those suitable for nucleic acid amplification reaction, which have been stabilised according to a method of the present invention.

According to another aspect this invention relates to a reaction vessel, particularly one suitable for conducting a nucleic acid amplification reaction, comprising reagents which have been stabilised according to a method of the present invention.

This invention also relates to the use of a reagent stabilised according to the present invention for conducting a nucleic acid amplification reaction.

According to another aspect this invention relates to a method of performing a nucleic acid amplification reaction comprising:
(i) preparing a reagent mixture according to the present invention;
(ii) adding to said reagent mixture the target material to be amplified, sufficient further magnesium ions to activate the amplification reaction and a suitable solvent; and
(iii) heating and cooling the so formed reaction mixture.

The amplification reaction is preferably a polymerase chain reaction and more preferably a real time polymerase chain reaction. The necessary reagents can be determined by one skilled in the art depending on the actual amplification reaction that is used. Most preferably the nucleic acid amplification reaction is a probe based real time PCR reaction.

The target nucleic acid is optionally added to the reagent mixture in aqueous solution, preferably in the volume of solution required to conduct the amplification reaction. Alternatively the magnesium ions may be added in aqueous solution, preferably in the volume of solution required to conduct the amplification reaction. Preferably the target nucleic acid and the magnesium ions are mixed together prior to addition to the reagent mixture. If neither the target material or the magnesium ions are added in solution, or are added in insufficient volume of solution for the amplification reaction to occur, it may be necessary to add further solvent, preferably water, to enable the reaction to proceed with the reagents at the desired concentration.

Optionally it may be necessary to purify or otherwise prepare the target material after it has been collected as a sample, for example a clinical sample or an environmental sample. Such preparation or purification can be conducted in any manner known in the art. These steps may include concentration of the target within a suitably small volume of solvent for the amplification reaction to occur.

After the solvent is added to the reagent mixture the dried reagents will reconstitute such that each of the necessary reagents is present in solution and at the desired and optimised concentration for the amplification to proceed.

It is necessary to add the additional magnesium ions to the reaction mixture in order that sufficient magnesium ions are available to activate the amplification reaction including to activate the polynucleotide polymerase. Furthermore it may have a role in buffering the reaction solution. The magnesium ions can be added by any suitable means. Preferably the target is dissolved in a prepared magnesium solution prior to addition to the dried reagents. This is ideal since being inorganic, magnesium salts need not be prepared or stored using special precautions against microbial contamination. Alternatively if the target material is to be eluted from the column that the column is designed such that magnesium ions are also eluted. Alternatively, if a layer of wax or grease is used when stabilising the dried reagent mixture, the magnesium compound may be contained within the layer of wax or grease. For example fatty acid salts of Mg are potentially soluble in oil/wax/grease and yet also extract into water when the oil/wax/grease contacts the hot water and therefore the magnesium can be stored in the oil/wax/grease layer. This means that as the reaction mixture is heated and the oil/wax/grease melts and floats to the top of the aqueous solution containing the target any magnesium present is released into the reaction mixture.

According to a still further aspect this invention relates to a method for stabilising reagents suitable for use in a nucleic acid amplification reaction comprising:
(i) preparing a reagent mixture comprising reagents suitable for use in a nucleic acid amplification reaction wherein the mixture comprises a polynucleotide polymerase;
(ii) drying the reagents; and
(iii) covering the dried reagents with a layer of wax or grease;
   characterised in that the reagent mixture comprises insufficient magnesium ions to activate an amplification reaction.

This method has the advantage of providing an improved method of stabilising a mixture of reagents, especially reagents suitable for use in a nucleic acid amplification reaction whilst allowing the reagent mixture to comprise a variety of different levels of magnesium ions, including very low levels of magnesium ions or alternatively no magnesium ions.

This invention also relates to reagents suitable for use in a nucleic acid amplification reaction which have been stabilised according to this method; a reaction vessel comprising reagents suitable for use in a nucleic acid amplification reaction which have been stabilised by a method according to this invention and also to a method of conducting a nucleic acid amplification reaction comprising taking reagents stabilised according to a method of the present invention, adding to the reagents a target nucleic acid and sufficient magnesium ions and heating the reaction mixture.

### EXAMPLES

The following examples further illustrate the preferred embodiments within the scope of the present invention. These examples are given solely for the purpose of illustration and are not to be construed as limitations of the present invention as many variations of the invention are possible without departing from its spirit or scope.

### Example 1

Real time PCR reactions were conducted using different concentrations of magnesium ions, supplied in the form of magnesium chloride, to determine the level of magnesium which could be added to a PCR reagent mixture without stimulating the activity of the thermostable polymerase enzyme.

The following PCR reagents were mixed to make a “2× master mix” which when diluted to a working concentration with distilled water comprised the following: 50 mM TRIZMA pH8.8, 200 μM dNTPs containing dUTP, 250 ng/μL BSA, 8% (v/v) glycerol, 0.02 U/μL uracil-N-glycosidase (UNG), 0.04 U/μL Taq polymerase, 0.03 μM TaqStart antibody.

To the above mixture various different concentrations of magnesium chloride were added (0 mM; 0.3 mM; 0.6 mM; 1 mM; 3 mM). In addition oligonucleotide primers (1 μM final concentration) and Sybr Gold dye (1:20000 dilution of stock) were also added. To half of the assays a target DNA was added to a concentration of approx. 1×10⁴ copies/μL per assay (t). The remainder of the assays were conducted in the presence of no target DNA material (ntc). This allowed for the clear identification of unwanted artefacts. The final assay volume in all cases was made up to 20 μL. Each assay was performed in duplicate.

The amplification was conducted in a glass capillary vessel in a Roche LightCycler and fluorescence data was collected, in the F1 channel, throughout each amplification. The following thermocycling conditions were used in all assays: 50° C. for 60 s; 95° C. for 60 s; 95° C. for 5 s; 60° C. for 5 s; 74° C. for 5 s. Heating at 95° C. for 5 s; 60° C. for 5 s; 74° C. for 5 s was then repeated over 50 cycles. At the end of cycle 50 the PCR reaction mixture was heated from 50° to 95° C. to generate melting peaks of the products.

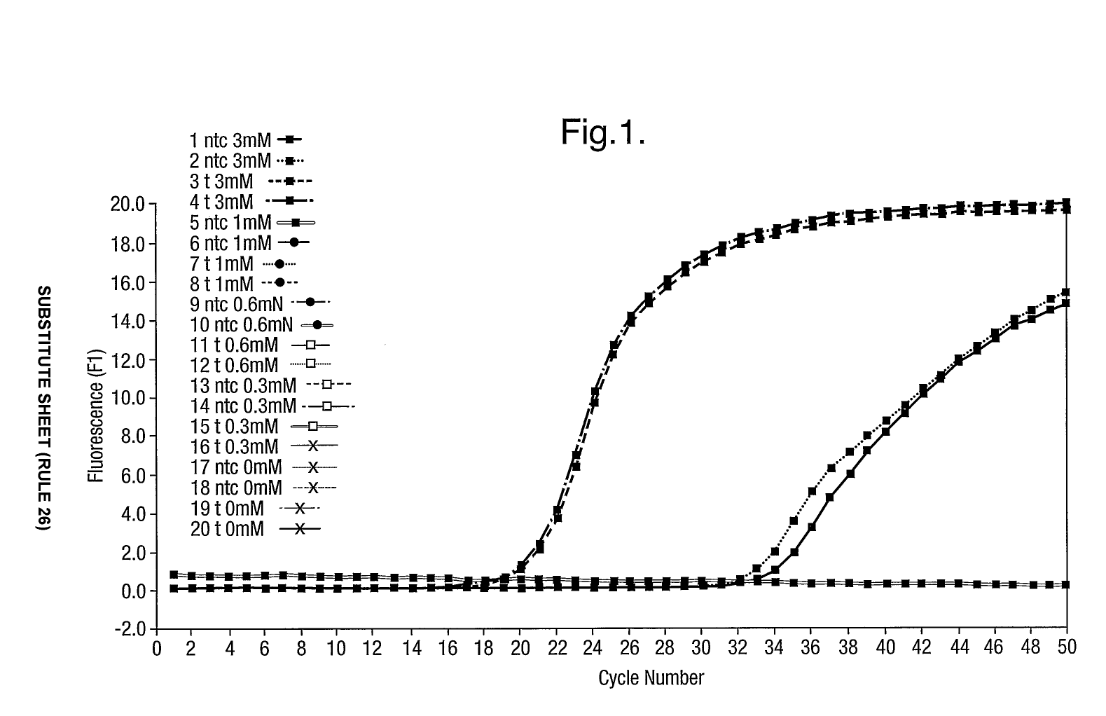

FIG. 1 shows the increase in fluorescence with cycle number as the amplification reaction proceeds.

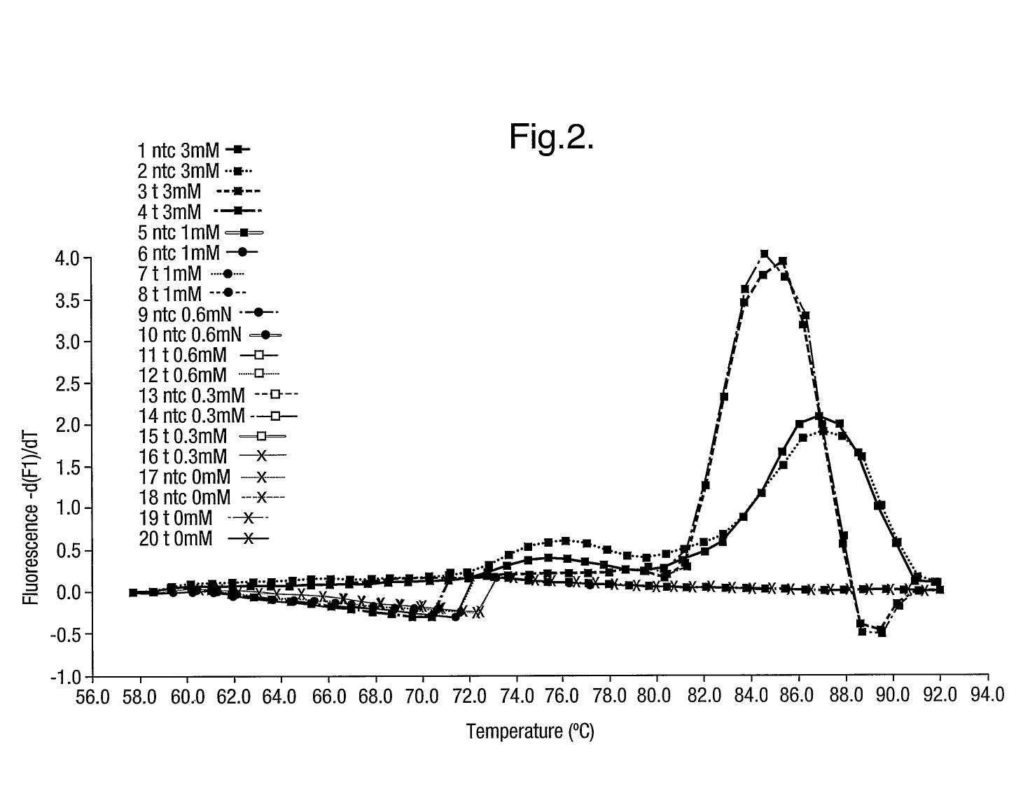

FIG. 2 shows the melting peaks of the products formed after amplification of each of the different assays.

The results indicated in FIG. 1 demonstrate that at concentrations of magnesium chloride in the range of 0 mM to 1 mM there was no increase in fluorescence over time indicating that there was no activity of the TAQ polymerase. However when the reaction was repeated at a concentration of 3 mM magnesium chloride there was an increase in fluorescence indicating that the TAQ polymerase was active and that amplification products were being formed. Even in the assays conducted at 3 mM magnesium chloride but in the absence of target DNA there was still an increase in fluorescence observed. This was as a result of unwanted artefacts and by-products forming from primer interactions and amplifications.

The results shown in FIG. 2 provide a melting peak analysis of the products formed from the amplification reactions conducted. As expected for those assays comprising less than 3 mM concentration of magnesium chloride no amplification products were formed and hence no peaks are observed. The assays conducted at 3 mM magnesium chloride comprising target DNA show a clean peak at about 83° C. This peak is indicative of the amplification product achieved by the amplification of the target.

However these results also indicate that when the assay was conducted with no target DNA added non specific artefacts were also formed. These are demonstrated by the broad peaks with a melting point higher and lower than that of the target product. However the presence of these non-specific artefacts further demonstrates the activity of the polymerase at concentrations of magnesium chloride at 3 mM.

Overall these results demonstrate that concentrations of MgCl₂, below the optimum required for a PCR assay, can be added to a liquid formulation and the resulting PCR will not generate any specific or non specific products. This further supports that even if these lower amounts of magnesium are added to a mixture of reagents during preparation of such a mixture prior to storage that there is unlikely to be any unwanted amplification occurring since the polymerase will be insufficiently activated.

### Example 2

Real time PCR reactions were conducted using reagents which had been prepared to contain different concentrations of magnesium ions, freeze dried and then stored. These assays were conducted to compare the effect on the nucleic acid amplification reaction of preparing the freeze dried reagents without magnesium or alternatively comprising a low level of magnesium chosen such that the polymerase was inactive.

Liquid formulations of PCR reagents were prepared as before to contain the following when reconstituted to a working concentration of 1×: 50 mM TRIZMA pH8.8, 200 μM dNTPs containing dUTP, 250 ng/μL BSA, 0.02 U/μL uracil-N-glycosidase (UNG), 0.04 U/μL Taq polymerase, 0.03 μM TaqStart antibody and 10% w/v trehalose.

These stock PCR reagent mixtures were then modified as follows such that they could be used in two different types of real time PCR amplification and detection reactions:
(i) dye binding assays wherein the reagent mixture additionally comprised oligonucleotide primers (1 μM final concentration) and Sybr Gold (1:20000 dilution of stock), with or without MgCl₂ added to concentrations of 300 μM or 3 mM; or
(ii) probe based assays, such as that described in WO 99/28500, wherein the reaction mixture additionally comprised oligonucleotide primers (1 μM final concentration), Sybr Gold (1:20000 dilution of stock) and Cy 5.5 labelled oligonucleotide probe with or without MgCl₂ at a concentration of 300 μM.

All assay formulations were then freeze dried into polypropylene PCR tubes in a condenser set at −60° C. and 600 mTorr according to the following thermal treatment process:
(i) sample is held at −50° C. for 2 mins;
(ii) sample ramped to −50° C. over 58 mins;
(iii) sample held at −50° C. for 120 mins;

The sample was then subjected to the following primary drying steps:
(i) sample held at −50° C. for 360 mins at 200 mTorr;
(ii) sample ramped to −20° C. over 60 mins at 200 mTorr;
(iii) sample held at −20° C. for 300 mins at 100 mTorr;
(iv) sample ramped to 20° C. over 80 mins at 50 mTorr;
(v) sample ramped to 20° C. over 400 mins at 50 mTorr;
(vi) sample held at 20° C. for 360 mins at 20 mTorr.

The sample was then stored at 25° C. until needed.

The tubes containing dried reagent were then reconstituted by the addition of purified water such that the assays could be performed. In half of the tubes the reconstitution mixture comprising target DNA at a concentration of 1×10⁴ copies/μL was added. In the other half of the tubes no DNA was added. To those tubes where magnesium had been added at a concentration of 300 μM prior to freeze drying a further 2.7 mM MgCl₂ was added. In those tubes which had contained no magnesium prior to freeze drying 3 mM MgCl₂ was added. In all cases the final reconstituted volume of the reagent mixture with or without target DNA was 20 μL. Sufficient materials were prepared that each assay could be repeated twice.

Each assay was then subjected to an amplification reaction as set out for example 1. At the end of cycle 50 the PCR reaction mixture was heated from 50° to 95° C. to generate melting peaks of the products.

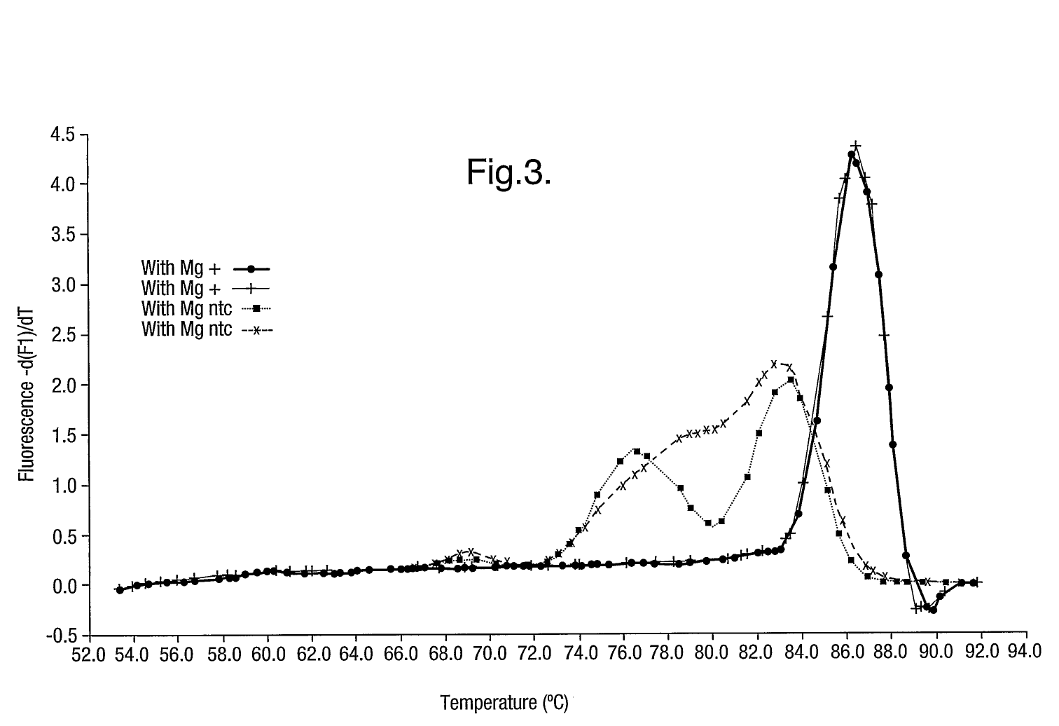

FIG. 3 shows the melting peaks of the products formed after amplification of the probe based assay wherein the reagents had been stored in the absence of magnesium chloride.

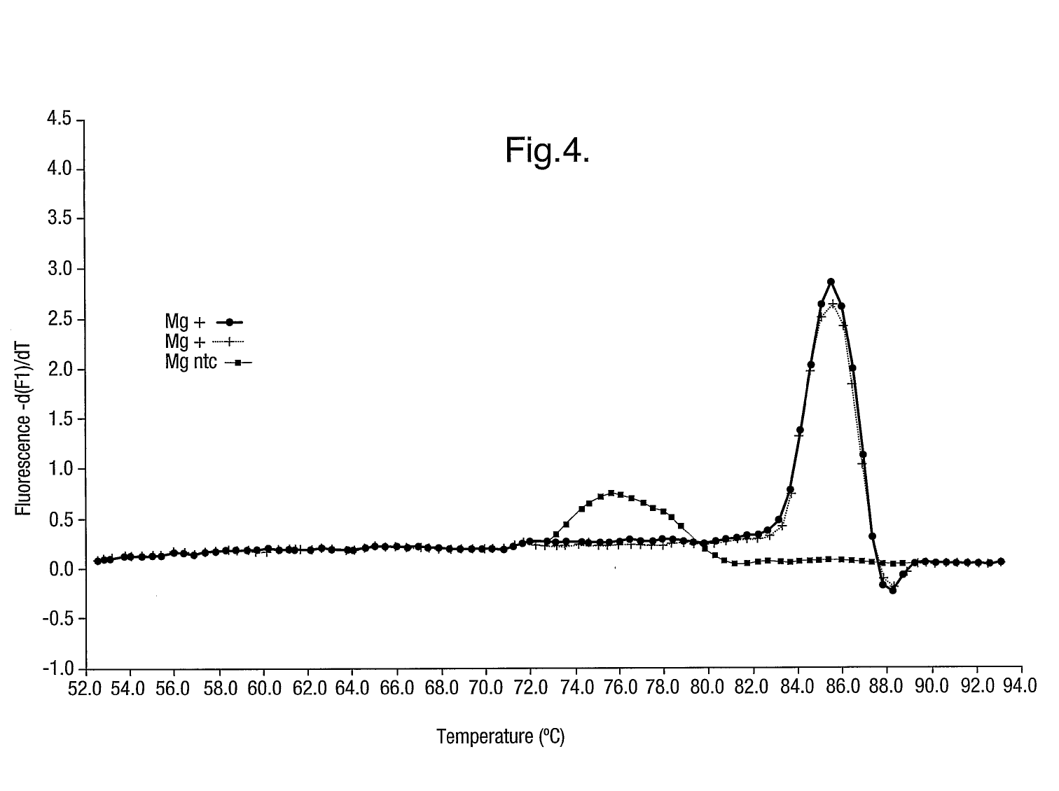

FIG. 4 shows the melting peaks of the products formed after amplification of the dye binding assay wherein the reagents had been stored in the presence of 300 μM magnesium chloride.

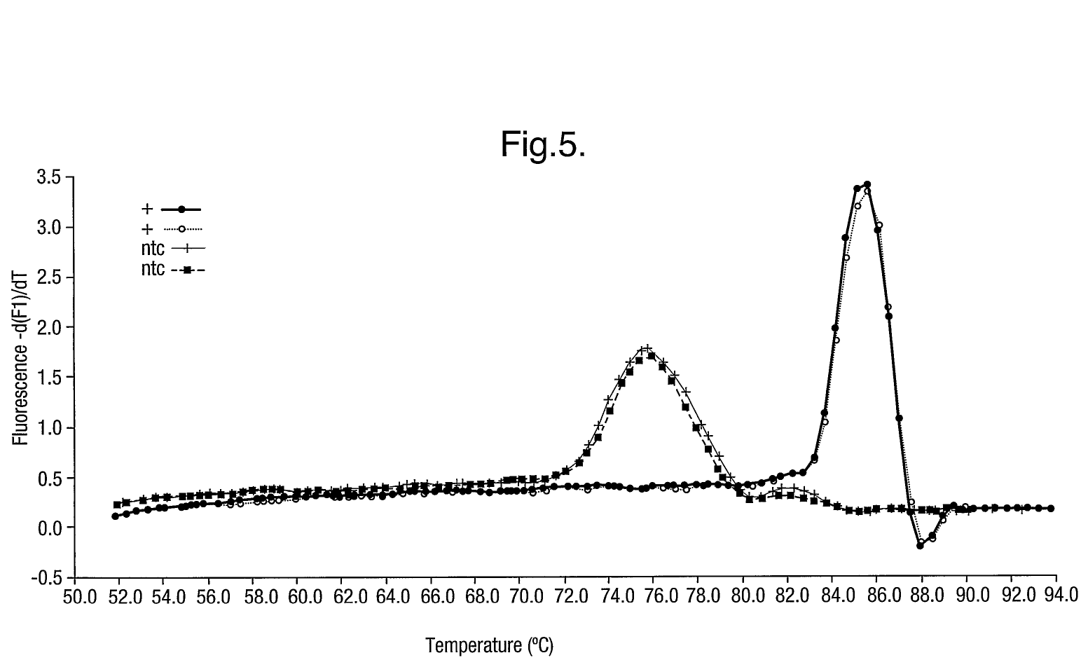

FIG. 5 shows the melting peaks of the products formed after amplification of the dye binding assay wherein the reagents had been stored in the presence of 3 mM magnesium chloride.

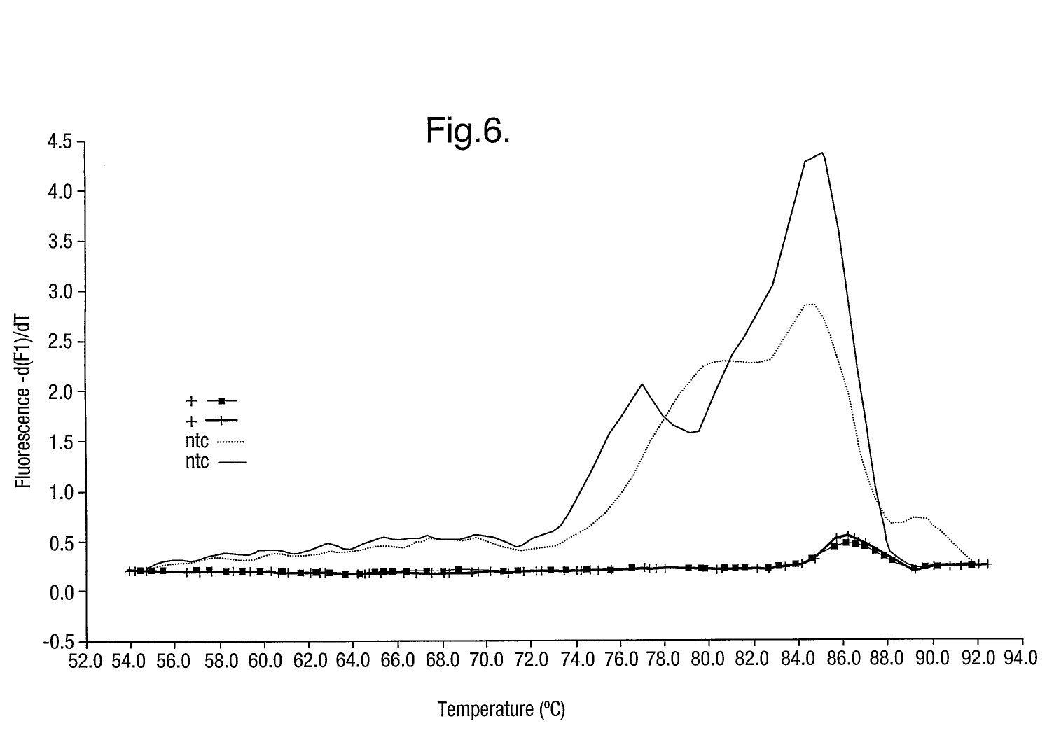

FIG. 6 shows the melting peaks of the products formed after amplification of the probe based assay wherein the reagents had been stored in the absence of magnesium chloride.

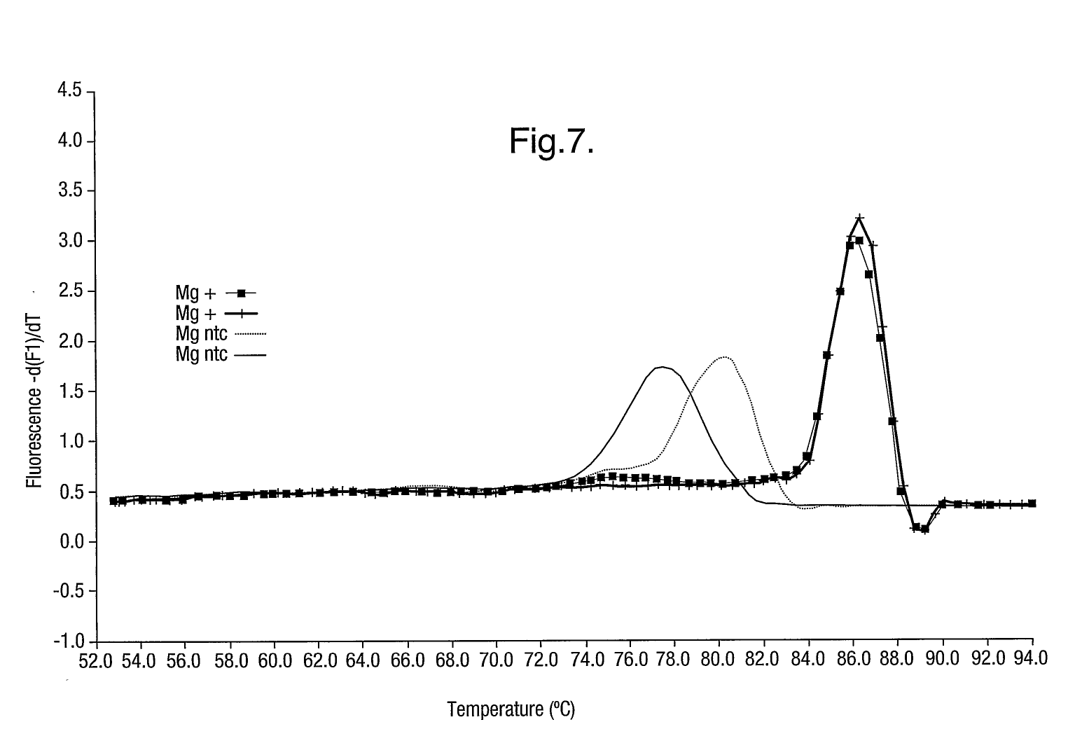

FIG. 7 shows the melting peaks of the products formed after amplification of the probe based assay wherein the reagents had been stored in the presence of 300 μM magnesium chloride.

The results shown in FIG. 3 demonstrate that when the dye binding assay is performed in the presence of target DNA using reagents which have been stored in the absence of magnesium chloride then only the desired amplification product is observed (peak at 86° C.). When the same assay is performed in the absence of any target DNA then a large amount of heterogeneous unwanted artefacts are produced as indicated by the broad peaks between 73° C. and 86° C.

The results shown in FIG. 4 demonstrate that when the dye binding assay is performed in the presence of target DNA using reagents which have been stored in the presence of a low concentration of magnesium (300 μM) the only product formed is the desired amplification product with a peak at 85° C. When the same assay is performed in the absence of target DNA then a small amount of unwanted artefacts are produced as indicated by the broad peak between 72° C. and 80° C.

The results shown in FIG. 5 demonstrate that when the dye binding assay is performed in the presence of target DNA using reagents which have been stored in the presence of high concentration of magnesium (3 mM) again the desired amplification product is observed with a peak at 85° C. When the same assay is performed in the absence of any target DNA then unwanted artefacts are produced as indicated by the broad peak between 72° C. and 84° C.

Comparing the results from FIGS. 3, 4 and 5 demonstrates that the concentration of magnesium chloride in the dried down reagents affects the quality of the products formed in the reconstituted dye binding assay. In all cases the amplification reaction in the presence of a excess amount of target DNA appears to proceed cleanly regardless of how the reagents are stored. However the difference in effect on the reaction in the presence of no target DNA is very interesting since this is likely to be indicative of how the reaction may proceed if only a very low concentration of target material is present as is often the case with clinical or environmental samples. Without wishing to be bound by theory it is believed that these results can be explained as follows. When the concentration of magnesium in the dried reagents is zero then a large and complex mixture of unwanted artefacts are formed which it is believed arise from primer interactions which occur during the freeze drying process and which can then act as a template for the polymerase enzyme when the reaction is reconstituted. When the concentration of magnesium in the dried reagents is low (300 μM) then the amount of unwanted artefacts observed when the reconstituted assay is performed is dramatically reduced indicative of a dramatic reduction in unwanted side reactions. It is believed that this occurs because the presence of some magnesium minimises any primer interactions and thereby minimises the formation of unwanted polymerase templates. However when the concentration of magnesium in the dried reagents is sufficient to provide polymerase activity (here 3 mM) then there is an increase in the level of unwanted artefacts again indicating some side reactions (although it should be noted that the amount of unwanted artefacts remained reduced and was cleaner than those seen in the absence of magnesium completely). Again it is believed that these artefacts form as a result of polymerase activity which occurs during the dry down of the reagents themselves. Overall these results demonstrate that in order to achieve a reduction in unwanted side reactions and therefore unwanted artefacts it is ideal to store the reagents in the presence of some magnesium but that the concentration is chosen to be sufficiently low such that the polymerase present in the reagent mixture is not active. This will both improve the efficiency of the amplification and the sensitivity of the test, especially when the target it only present at a very low concentration.

FIGS. 6 and 7 relate to probe based assays.

The results shown in FIG. 6 demonstrate that when the probe based assay is performed using reagents which have been stored in the absence of magnesium and there has been no target material added to the assay that a very large amount of heterogeneous unwanted artefacts are produced, indicated by the large and very broad peak between 73° C. and 90° C. Alternatively when the same assay is performed in the presence of target DNA, although no unwanted artefacts are seen on these graphs, the efficiency of the amplification is very low and only a very small amount of amplified target is produced, indicated by the very small peak at 85° C.

The results shown in FIG. 7 demonstrate that when the probe based assay is performed in the presence of target DNA using reagents which have been stored in the presence of a low concentration of magnesium chloride (300 μM) the assay is very clean and the only product formed is the desired amplification product with a peak at 85° C. When the same assay is performed in the absence of any target DNA then unwanted artefacts are again seen as indicated by the peaks between 74° C. and 82° C. However, the amount of unwanted artefacts produced is significantly lower than that observed in assays reconstituted from dried reagents containing no magnesium chloride.

Comparing the results in FIGS. 6 and 7 it can be seen that when the assay is conducted in the presence of a target DNA with magnesium prepared reagents, significantly more desired product is formed. Furthermore when the probe based assay is conducted in the absence of a target DNA there is a dramatic reduction in the amount of unwanted artefacts produced when using magnesium prepared reagents vs those prepared and stored in the absence of magnesium. Again it is important to reduce the level of unwanted artefacts such that the reaction efficiency and sensitivity is increased for samples containing only a very low concentration of target material such is often the case in a clinical or environmental sample. Without wishing to be bound by theory it is believed that the reduction of unwanted artefacts occurs because the presence of a low level of magnesium ions acts to minimise interaction between the oligonucleotides during the drying process thereby reducing or eliminating their interaction during subsequent amplification.

Comparing the results from the two different types of PCR assays conducted it can be seen that both responded positively when the reagents used were those prepared using a low concentration of magnesium, ie there was a reduction in the amount of unwanted artefacts produced. It can also be seen that with respect to the amplification of target the probe based assay also responded positively in the increase in the production of desired target achieved. These results clearly demonstrate that preparation and storage of the reagents in the presence of magnesium ions not only provides a stable method for the stabilisation of the reagents suitable but also optimises the reagents such that the amplification reaction is both more sensitive and more efficient.

1. A method for stabilising reagents suitable for use in a nucleic acid amplification reaction comprising:
(i) preparing a reagent mixture comprising reagents suitable for use in a nucleic acid amplification reaction wherein the mixture comprises a polynucleotide polymerase; and
(ii) drying the reagents;
characterised in that said reagent mixture comprises from about 0.1% to about 50% of the final concentration of magnesium ions required to activate an amplification reaction.

2. A method according to claim 1 wherein the nucleic acid amplification reagent mixture comprises one or more reagents selected from the group consisting of oligonucleotide primers, deoxyribonucleoside triphosphates, ribonucleoside triphosphates, oligonucleotide probes, intercalating fluorescent dyes, bovine serum albumin, internal control nucleic acid and mixtures thereof.

3. A method according to claim 2 wherein the nucleic acid amplification reagent mixture comprises oligonucleotide primers, deoxyribonucleoside triphosphates, and buffer.

4. A method according to claim 1 wherein the reagent mixture is dried using either a freeze drying method or a lyophilisation method.

5. A method according to claim 1 wherein the reagent mixture comprises from about 3% to about 30% and more preferably from about 5% to about 15% of the final concentration of magnesium ions.

6. A method according to claim 1 wherein after drying the reagent mixture a covering of wax or grease is added.

7. A method according to claim 6 wherein the wax or grease is in contact with the reagents.

8. A method according to claim 6 wherein the wax or grease forms a plug within a vessel above the reagents.

9. A method according to claim 6 wherein the wax or grease has a melting point in the range of from about 40° C. to about 90° C.

10. Reagents suitable for use in a nucleic acid amplification reaction stabilised according to a method according to claim 1.

11. A reaction vessel suitable for use in a nucleic acid amplification reaction comprising reagents stabilised according a method according to claim 1.

12. Use of reagents prepared according to claim 1 in a nucleic acid amplification reaction.

13. A method of performing a nucleic acid amplification reaction comprising:
(i) preparing a reagent mixture according to claim 1;
(ii) adding to said reagent mixture the target material to be amplified, sufficient further magnesium ions to activate the amplification reaction and a suitable solvent; and
(iii) heating and cooling the so formed reaction mixture.

14. A method according to claim 13 wherein the target material is added as an aqueous solution.

15. A method according to claim 14 wherein the further magnesium ions are added in conjunction with the target material.

16. A method according to claim 13 wherein after drying the reagent mixture the dried reagents are covered with a layer of wax or grease.

17. A method according to claim 16 wherein the further magnesium ions are contained within the layer of wax or grease.

18. A method for stabilising reagents suitable for use in a nucleic acid amplification reaction comprising:
(i) preparing a reagent mixture comprising reagents suitable for use in a nucleic acid amplification reaction wherein the mixture comprises a polynucleotide polymerase;
(ii) drying the reagents; and
(iii) covering the dried reagents with a layer of wax or grease;
characterised in that the reagent mixture comprises insufficient magnesium ions to activate an amplification reaction.
